(19) United States
(12) Patent Application Publication
(10) Pub. No.: US 2011/0275685 A1
(43) Pub. Date: Nov. 10, 2011

(54) SALT AND POLYMORPHS OF A KINESIN INHIBITOR COMPOUND

(76) Inventors: Michael Mutz, Freilburg (DE); Jean-Louis Reber, Kembs (FR); Stephanie Monnier, Senlis (FR)

(21) Appl. No.: 13/144,558

(22) PCT Filed: Jan. 25, 2010
(86) PCT No.: PCT/EP10/50770

§ 371 Date: Jul. 14, 2011

(30) Foreign Application Priority Data
Jan. 26, 2009 (EP) 09151344.0

(51) Int. Cl. A61K 31/4164; C07D 233/58

(52) U.S. Cl. 514/400; 548/338.1

(57) ABSTRACT

The present invention relates to salts, polymorphs and hydrates of the kinesin inhibitor compound N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide, processes to prepare such salts, polymorphs and hydrates and a liquid formulation comprising at least one of these salts, polymorphs and hydrates.

The present invention relates to salts and polymorphs of the kinesin inhibitor compound N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide.

Kinesins are motor proteins that hydrolyze adenosine triphosphate as they travel along microtubules and generate mechanical force. These proteins are characterized by containing a motor domain having about 350 amino acid residues. The crystal structures of several kinesin motor domains have been resolved.

Currently, about one hundred kinesin-related proteins (KRP) have been identified. Kinesins are involved in a variety of cell biological processes including transport of organelles and vesicles, and maintenance of the endoplasmic reticulum. Several KRP's interact with the microtubules of the mitotic spindle or with the chromosomes directly and appear to play a pivotal role during the mitotic stages of the cell cycle. These mitotic KRP's are of particular interest for the development of cancer therapeutics.

Kinesin spindle protein (KSP) (also known as Eg5, HsEg5, KNSL1, or KIF11) is one of several kinesin-like motor proteins that are localized to the mitotic spindle and known to be required for formation and/or function of the bipolar mitotic spindle.

In 1995, the depletion of KSP using an antibody directed against the C-terminus of KSP was shown to arrest HeLa cells in mitosis with monoastral microtubule arrays (Slangy et al., Cell 83:1159-1169, 1995). Mutations in bimC and cut7 genes, which are considered to be homologues of KSP, cause failure in centrosome separation in Aspergillus nidulans (Enos. A. P., and N. R. Morris, Cell 60:1019-1027, 1990) and Schizosaccharomyces pombe (Hagan, I. and M. Yanagida, Nature 347:563-566, 1990). Treatment of cells with either ATRA (all trans-retinoic acid), which reduces KSP expression on the protein level, or depletion of KSP using antisense oligonucleotides revealed a significant growth inhibition in DAN-G pancreatic carcinoma cells indicating that KSP might be involved in the antiproliferative action of all trans-retinoic acid (Kaiser, A., et al., J. Biol. Chem. 274, 18925-18931, 1999). Interestingly, the Xenopus laevis Aurora-related protein kinase pEg2 was shown to associate and phosphorylate XlEg5 (Giet, R. et al., J. Biol. 274:15005-15013, 1999). Potential substrates of Aurora-related kinases are of particular interest for cancer drug development. For example, Aurora 1 and 2 kinases are over expressed on the protein and RNA level and the genes are amplified in colon cancer patients.

The first cell permeable small molecule inhibitor for KSP, “monoastral,” was shown to arrest cells with monopolar spindles without affecting microtubule polymerization as do conventional chemotherapeutics such as taxanes and vinca alkaloids (Mayer, T. U, et al., Science 286:971-974, 1999), Monastrol was identified as an inhibitor in phenotype-based screens and it was suggested that this compound may serve as a lead for the development of anticancer drugs. The inhibition was determined not to be competitive with respect to adenosine triphosphate interaction with KSP, and was found to be rapidly reversible (DeBonis. S., et al., Biochemistry 42:338-349, 2003; Kapoor, T. M., et al., J. Cell Biol. 150:975-988, 2000).

In light of the importance of improved chemotherapeutics, there is a need for KSP inhibitors that are effective in vivo inhibitors of KSP and KSP-related proteins. Some inhibitors of KSP have been reported previously. For example, WO 2006/002236 and PCT/US2006/031129 disclose certain classes of compounds indicated to be inhibitors of KSP.

A specific kinesin inhibitor compound is N-((S)-3-amino-4-fluorobutyl)-N-((R)-1-(1-benzyl-4-(2,5-difluorophenyl)-1H-imidazol-2-yl)-2,2-dimethylpropyl)-2-hydroxyacetamide which has the following chemical structure:

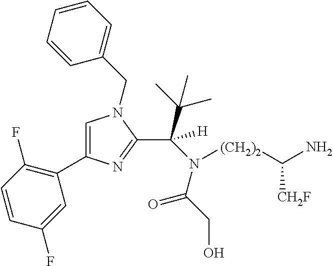

However, after a specific compound is identified as a promising candidate for use in a pharmaceutical composition, it is still necessary to fine-tune its properties with respect to a number of critical parameters, such as stability in solid state and/or liquid formulations, hygroscopicity, crystallinity, toxicological considerations, melting point, or solubility in water and aqueous media.

Relevant properties of a pharmaceutical compound are affected by the type of salt and/or crystalline modification. The criteria for the selection of the salt or the crystalline modification depend inter alia on the planned route(s) of administration.

It has now been surprisingly found that under certain conditions new salts of N-((S)-3-amino-4-fluorobutyl)-N-((R)-1-(1-benzyl-4-(2,5-difluorophenyl)-1H-imidazol-2-yl)-2,2-dimethyl-propyl)-2-hydroxyacetamide can be provided which have advantageous utilities and properties.

Additionally, it has been surprisingly found that under certain conditions new solid forms of N-((S)-3-amino-4-fluorobutyl)-N-((R)-1-(1-benzyl-4-(2,5-difluorophenyl)-1H-imidazol-2-yl)-2,2-dimethylpropyl)-2-hydroxyacetamide can be provided which are described hereinafter as crystalline forms D and H and amorphous form A, and which have advantageous utilities and properties.

Thus, according to a first aspect the present invention provides a salt of N-((S)-3-amino-4-fluorobutyl)-N-((R)-1-(1-benzlyl-4(2,5-difluorophenyl)-1H-imidazol-2-yl)-2,2-dimethylpropyl)-2-hydroxyacetamide, wherein the anion is selected from the group consisting of mesylate, tosylate, hippurate, glycolate, and sulfate.

In a preferred embodiment, the anion is mesylate and the salt is present as a hydrate.

In the context of the present invention, the term “hydrate” is to be interpreted according to its commonly accepted meaning. It refers to water molecules incorporated into the crystal structure of a host compound and encompasses stoichiometric as well as non-stoichiometric hydrates.

Preferably, the hydrate of the mesylate salt contains water in an amount of 1 to 6 wt % determined by thermogravimetric analysis.

Preferably, the hydrate of the mesylate salt is a hemihydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×0.5 H₂O), a monohydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×1 H₂O), sesquihydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×1.5 H₂O) or a dihydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×2 H₂O).

According to a further aspect, the present invention provides a process for the preparation of the salts as defined above, wherein the salt is precipitated from a polar solvent system.

The salts of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide as described above can be prepared by precipitation methods known to the skilled person.

The starting compound N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide from which the salts are prepared can be obtained by methods known in the art and further described below. For example, methods for making the compound are described in PCT/US2005/022062 (WO 2006/002236) and the corresponding U.S. patent applications. Examples of additional synthesis methods applicable to the preparation of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide are provided in the reaction schemes below.

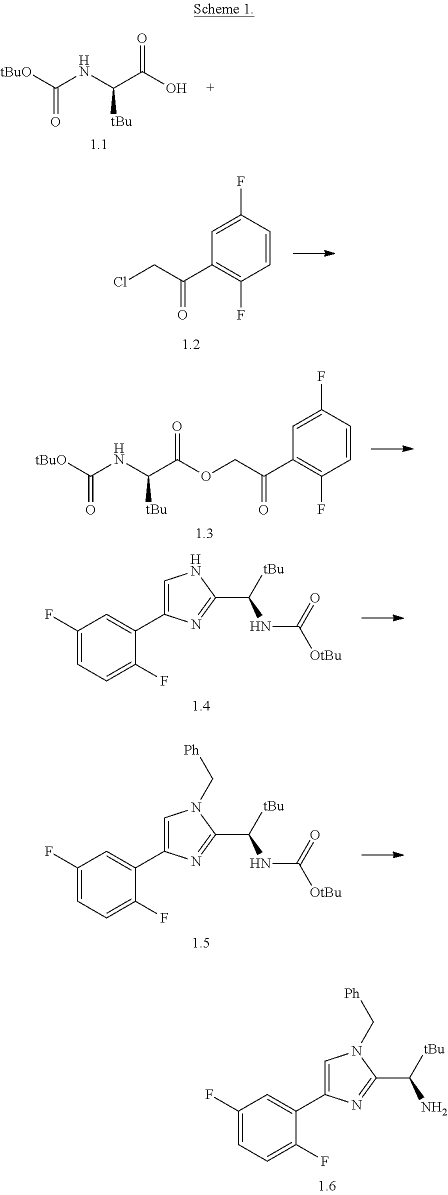

Compound 1.1 and 1.2 were reacted with K₂CO₃ in acetone containing Kl. The use of K₂CO₃/acetone was found to be superior to Cs₂CO₃/ethanol because of the lower cost of K₂CO₃ and because compound 1.3 precipitated from the acetone solution upon addition of water, removing the need for an aqueous workup to extract 1.3. Keto ester 1.3 was then refluxed with ammonium acetate (NH₄OAc) in toluene to give imidazole 1.4. The use of toluene was found to afford higher yields of the imidazole in comparison to refluxing in xylenes with a Dean Stark trap, as the latter method led to the removal of ammonium acetate from the reaction mixture into the trap. Reaction of 1.4 with benzylbromide and K₂CO₃ in dimethylformamide afforded 1.5, which can be precipitated from the reaction solution upon addition of water. Treatment of 1.5 with methanol and acetyl chloride gave the HCl salt of 1.6 which was then converted to its free base when titrated with a NaOH/methanol solution. The formation of 1.6 from 1.1 and 1.2 was found to proceed with 81% yield with high purity (>97% as determined by HPLC) and high optical purity (>99% e.e.).

Scheme 2 illustrates the preparation of an aldehyde that can be used in the reductive amination step to prepare N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide,

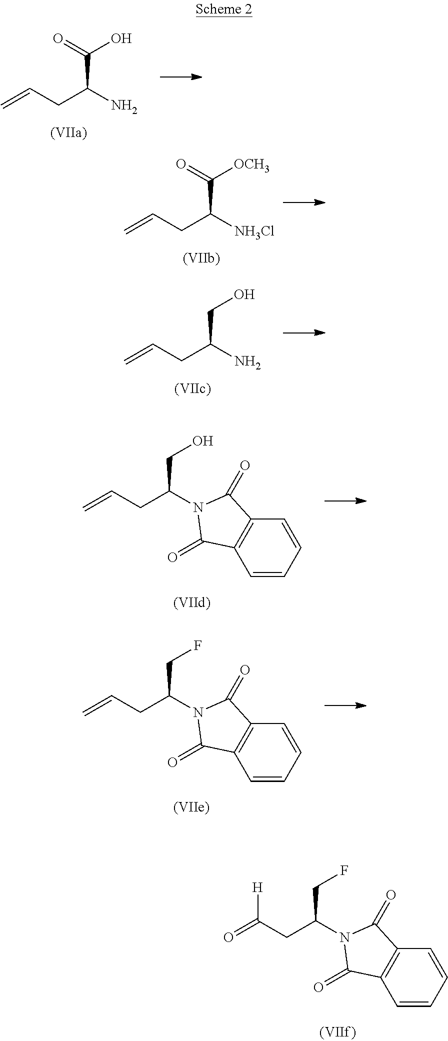

After the reductive amination, known acylating agents and conditions are used to acylate the secondary amine to provide the starting compound N-((S-3-amino-4-fluorobutyl)-N-((R)-1-(1-benzlyl-4-(2,5-difluorophenyl)-1H-imidazol-2-yl)-2,2-dimethylpropyl)-2-hydroxyacetamide, Scheme 3 illustrates the reductive amination. Acylation of the amine followed by deprotection of the phthalimide and removal of a protecting group on the free hydroxyl group provides N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide. Suitable protective groups for the hydroxyl include, for example, benzyl ethers that can be removed by hydrogenolysis and alkyl carbonates that can be selectively removed with reagents such as trimethylsilyl iodide.

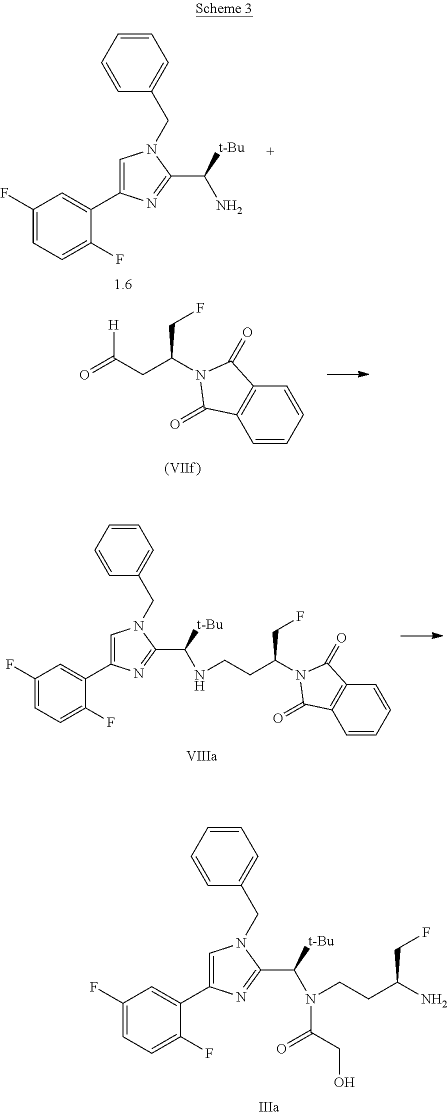

As indicated above, the salts of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide described above can be prepared by precipitation methods which are in principle known to the skilled person.

Preferably, the polar solvent system comprises a polar solvent selected from an alcohol, preferably isopropanol and/or tert-butanol, a nitrile, preferably acetonitrile, acetone, or any mixture thereof. The polar solvent system may further comprise water.

For intravenous (i.v.) applications, the salt as described above needs to be provided in liquid formulations of sufficient stability.

Thus, according to a further aspect, the present invention provides a liquid formulation, prepared by dissolving or suspending the salt as defined above in a solvent or suspension medium. Preferably, the salt employed is the hydrate form D or hydrate form H as further defined below.

Preferably, the solvent or suspension medium is water having a pH of less than 7, more preferably a pH of 6 or less, even more preferably a pH of 5.5 or less, optionally further comprising an additional pharmaceutically acceptable organic solvent dissolving in water.

As indicated above, relevant properties of a pharmaceutical compound such as stability in solid state, hygroscopicity, processability, solubility etc. can be affected by the type of crystalline modification.

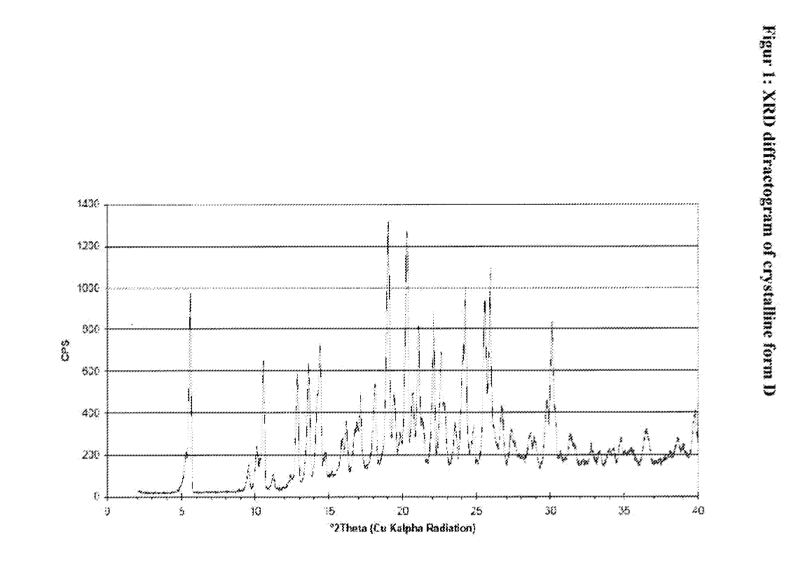

According to a further aspect, the present invention provides a crystalline hydrate form D of N-((S)-3-amino-4-fluorobutyl)-N-((R)-1-(1-benzlyl-4(2,5-difluorophenyl)-1H-imidazol-2-yl)-2,2-dimethylpropyl)-2-hydroxyacetamide. In a preferred embodiment, the form D shows the X-ray diffraction diagram indicated in FIG. 1.

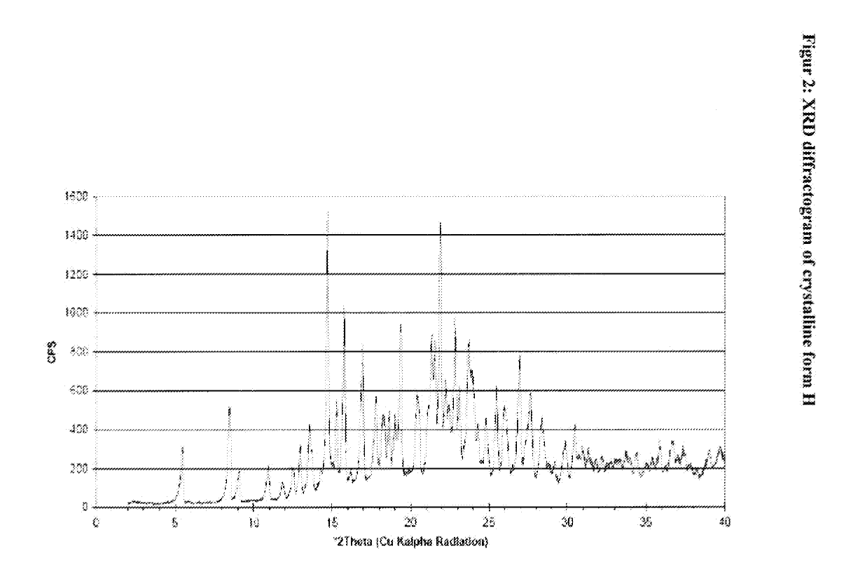

According to a further aspect, the present invention provides a crystalline hydrate form H of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide. In a preferred embodiment, the form H shows the X-ray diffraction diagram indicated in FIG. 2.

Preferably, the crystalline form H is prepared by crystallization in acetonitrile, preferably dried acetonitrile, and subsequent isolation in humid air, for instance as described in the Examples.

Preferably, the crystalline form D is prepared in an acetonitrile/water solvent system, for instance as described in the Examples.

According to a further aspect, the present invention provides a pharmaceutical composition comprising an active component which is selected from the salt as defined above.

Preferably, the pharmaceutical composition is used for the treatment of a proliferative disease such as cancer.

More preferably, the proliferative disease is selected from a solid tumor or a hematological cancer in a mammal

Preferably, the solid tumor solid tumor is selected from the group consisting of lung carcinoma, breast carcinoma, ovarian carcinoma, skin carcinoma, colon carcinoma, urinary bladder carcinoma, liver carcinoma, gastric carcinoma, prostate cancer, renal cell carcinoma, nasopharyngeal carcinoma, squamous cell carcinoma, thyroid papillary carcinoma, cervical carcinoma, small cell lung carcinoma (SCLC), non-small cell lung carcinoma, pancreatic cancer, head and neck squamous cell cancer and sarcomas.

The present invention is now described in further detail by the examples provided below.

### EXAMPLES

X-ray diffractograms of crystalline forms were measured with a PANalytical X'Pert powder diffractometer (Copper Kalpha radiation).

### Example 1

### Tosylate Salt

The tosylate salt of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide was prepared in acetonitrile as solvent.

The tosylate salt has a melting temperature T (measured by DSC as melting onset) of 207° C.

The weight loss (%) on drying, measured by thermogravimetry, of the salt as prepared was 1.12%. If kept for 1 day at 92% relative humidity, the weight loss was 1.71%. This clearly indicates that the tosylate salt is of low hygroscopicity.

### Example 2

### Mesylate Salt Amorphous Form

The mesylate salt of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide is prepared in acetonitrile as solvent.

The weight loss (%) on drying, measured by thermogravimetry, of the salt as prepared was 2.08%. If kept for 1 day at 92% relative humidity, the weight loss was 2.21%. This clearly indicates that the mesylate salt is of low hygroscopicity.

### Example 3

### Hippurate Salt

The hippurate salt of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide was prepared in an acetone/water solvent system. The hippurate salt has a melting temperature Tm (measured by DSC as melting onset) of 80° C.

The weight loss (%) on drying, measured by thermogravimetry, of the salt as prepared was 1.76%. If kept for 1 day at 92% relative humidity, the weight loss was 2.14%. This clearly indicates that the hippurate salt is of low hygroscopicity.

### Example 4

### Sulfate Salt

The sulfate salt of N-((S)-3-amino-4-fluorobutyl)-N-((R)-1-(1-benztyl-4-(2,5-difluorophenyl)-1H-imidazol-2-yl)-2,2-dimethylpropyl)-2-hydroxyacetamide was prepared in isopropanol as solvent.

The sulfate salt has a melting temperature Tm (measured by DSC as melting onset) of 98° C.

### Example 5

### Glycolate Salt

The glycolate salt of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide was prepared in isopropanol as solvent.

### Example 6

### Stability of the Salts in the Solid State

Tests for solid state stability were made for the mesylate, tosylate and hippurate salts as prepared above. The results are shown below in Table 1.

TABLE 1
Solid state stability

| | Salt Form | | | | | |
|---|---|---|---|---|---|---|
| | Tosylate | | Mesylate | | Hippurate | |
| | Amount of | | Amount of | | Amount of | |
| | degra- | | degra- | | degra- | |
| Test | dation | | dation | | dation | |
| Con- | products | | products | | products | |
| ditions | [%] | Colour | [%] | Colour | [%] | Colour |

2 weeks, 50° C., tight container

| | | | | | | |
|---|---|---|---|---|---|---|
| Bulk | 0.15 | A | 0.07 | A | 0.79 | A |
| (HPLC) | | | | | | |

2 weeks, 50° C., 75% r.h.

| | | | | | | |
|---|---|---|---|---|---|---|
| Bulk | 0.19 | A | <0.05 | A | 1.02 | A |
| (HPLC) | | | | | | |

Xenon light (approx. 1200 kLuxh)

| | | | | | | |
|---|---|---|---|---|---|---|
| Bulk | 0.05 | A | <0.05 | A | 0.43 | A |
| (HPLC) | | | | | | |

Colour:
A: No change of colour;
B: Slight discolouration;
C: Medium discolouration;
D: Strong discolouration

The results of Table 1 clearly indicate that the salts are the in the solid state.

### Example 7

### Preparation of Liquid Formulations by Dissolving/Suspending the Salts

For intravenous (i.v.) applications, stability of the salts in liquid formulations is relevant as well.

From the mesylate, tosylate and hippurate salts as prepared above, liquid formulations were prepared by dissolving/suspending these salts in a solvent/suspension medium. Stability tests were made on these liquid formulations. The amount of degradation products and the degree of discoloration were established. The results are shown below in Table 2.

TABLE 2
Stability of liquid formulations

| | Salt Form | | | | | |
|---|---|---|---|---|---|---|
| | Tosylate | | Mesylate | | Hippurate | |
| | Amount of | | Amount of | | Amount of | |
| | degra- | | degra- | | degra- | |
| Test | dation | | dation | | dation | |
| Con- | products | | products | | products | |
| ditions | [%] | Colour | [%] | Colour | [%] | Colour |

0.1% Solutions or suspensions, 1 week, 50° C.

| | | | | | | |
|---|---|---|---|---|---|---|
| pH 3 | 0.27 | A | 0.25 | A | 0.48 | A |
| pH 4.5 | 0.59 | A | 0.69 | A | 1.03 | A |
| pH 5 | 1.81 | A | 0.71 | A | 1.80 | A |
| pH 7 | 7.60 | A | 3.87 | A | 5.32 | A |
| Metha- | 0.35 | A | 0.21 | A | 11.81 | A |
| nol | | | | | | |

2% Solutions or suspensions, 1 day, room temperature

| | | | | | | |
|---|---|---|---|---|---|---|
| DMSO | 0.14 | A | 0.07 | A | 0.78 | A |
| Di- | 0.33 | A | 0.19 | A | 0.82 | A |
| lution, | | | | | | |
| pH | | | | | | |
| 6.8⁽¹⁾ | | | | | | |

5% Solutions, 1 day, room temperature

| | | | | | | |
|---|---|---|---|---|---|---|
| DMSO | 0.15 | A | <0.05 | A | 0.94 | A |
| Di- | 0.11 | A | 0.30 | A | 0.84 | A |
| lution, | | | | | | |
| pH 6.8 | | | | | | |

⁽¹⁾A portion of the freshly prepared solution/suspension was diluted with a buffer pH 6.8 in the ratio 1:100.
Colour:
A. No change of colour;
B: Slight discolouration;
C: Medium discolouration;
D: Strong discolouration

If the pH of the solvent system is below 7, stability can be kept on a sufficiently high level.

In methanol, the mesylate and tosylate salts are very stable. In DMSO, either diluted or not, mesylate, tosylate and hippurate salts are very stable.

### Example 8

### Preparation of crystalline form D of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate

2.40 g of amorphous N-((S)-3-amino-4-fluorobutyl)-N-((R)-1-(1-benzlyl-4-(2,5-difluoro-phenyl)-1H-imidazol-2-yl)-2,2-dimethylpropyl)-2-hydroxyacetamide were dissolved at 50° C. in 9 ml acetonitrile/water 95:5 v/v having a water activity of 0.6, and stirred. Within about 1 minute, a suspension was formed. Another 6 ml of acetonitrile/water 95:5 v/v were added. Subsequently, the suspension was cooled to room temperature in about 1 hour and stirred for 14 hours. Then, the suspension was filtered and air dried for about 5 minutes. Yield: 1.82 g (75%). The obtained material was analyzed by X-ray diffraction (FIG. 1). The solubility of the obtained material in water is 4.4 mg/ml, in buffer with pH 6.8 it is 0.010 mg/ml and in 0.1 N HCl it is 1.4 mg/ml.

### Example 9

### Preparation of crystalline form H of N-((S)-3-amino-4-fluorobutyl)-N-((R)-1-(1-benzyl-4-(2,5-difluorophenyl)-1H-imidazol-2-yl)-2,2-dimethylpropyl)-2-hydroxyacetamide mesylate

123 mg of amorphous N-((S)-3-amino-4-fluorobutyl)-N-((R)-1-(1-benzlyl-4-(2,5-difluoro-phenyl)-1H-imidazol-2-yl)-2,2-dimethylpropyl)-2-hydroxyacetamide were dissolved in 0.5 ml methanol/water (1:1, v:v) and stirred for one day at room temperature. The obtained suspension was filtered and dried at the air for approximately 2 minutes. The obtained material was analyzed by X-ray diffraction (FIG. 2).

1. A salt of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide, wherein the anion is selected from the group consisting of mesylate, tosylate, hippurate, glycolate, and sulfate.

2. The salt according to claim 1, wherein the anion is mesylate and the salt is a hydrate.

3. The salt according to claim 2, wherein the hydrate is a hemihydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×0.5 H₂O), a monohydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×1 H₂O), sesquihydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×1.5 H₂O) or a dihydrate (N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate×2 H₂O).

4. A crystalline hydrate form D of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate.

5. A crystalline hydrate form H of N-((S)-3-amino-4-fluorobutyl)-N-((R)-1-(1-benzlyl-4-(2,5-difluorophenyl)-1H-imidazol-2-yl)-2,2-dimethylpropyl)-2-hydroxyacetamide mesylate.

6. A process for the preparation of one of the crystalline form H according to claim 5, wherein such form is prepared by crystallization in acetonitrile and subsequent isolation in humid air.

7. A liquid formulation, prepared by dissolving or suspending at least one salt according to claim 1 in a solvent or suspension medium, which comprises water having a pH of less than 7.
